(19) United States
(12) Patent Application Publication
(10) Pub. No.: US 2010/0069948 A1
(43) Pub. Date: Mar. 18, 2010

(54) SELF-EXPANDABLE ANEURYSM FILLING DEVICE, SYSTEM AND METHOD OF PLACEMENT

(75) Inventors: Erol Veznedaroglu, Philadelphia, PA (US); Marcelino Gorospe, Redwood City, CA (US); Edsel San Diego, San Jose, CA (US)

Correspondence Address: FULWIDER PATTON LLP, HOWARD HUGHES CENTER, 6060 CENTER DRIVE, TENTH FLOOR, LOS ANGELES, CA, 90045, US

(73) Assignee: MICRUS ENDOVASCULAR CORPORATION, San Jose, CA (US)

(21) Appl. No.: 12/558,466

(22) Filed: Sep. 11, 2009

Related U.S. Application Data
(60) Provisional application No. 61/096,546, filed on Sep. 12, 2008.

(51) Int. Cl. A61M 29/00

(52) U.S. Cl. 606/194

(57) ABSTRACT

The self-expandable aneurysm filling device, system and method provide for placement of the stent into an aneurysm to at least partially fill and stabilize the aneurysm. The self-expandable aneurysm filling device has a compressed undeployed configuration and an expanded three-dimensional deployed configuration, and a severable deployment junction releasably connects the self-expandable aneurysm filling device to a pusher wire. The severable deployment junction can be mechanically, electrolytically, or thermally severed to separate the self-expandable aneurysm filling device from the pusher wire.

### CROSS-REFERENCES TO RELATED APPLICATIONS

This application is based upon U.S. Provisional Application No. 61/096,546, filed Sep. 12, 2008, which is incorporated by reference in its entirety.

### BACKGROUND OF THE INVENTION

The present invention relates generally to endovascular devices. More specifically, the present invention relates to an endovascular device for filling of a vascular pathology such as an intracranial aneurysm.

Current treatment of cerebral aneurysms is performed by either an open surgical clipping of the aneurysm or by an interventional endovascular route. The mainstay of the interventional endovascular treatment involves the placement of one or more coils within the aneurismal sac via a microcatheter. One of the limitations associated with interventional endovascular therapy is that “wide-necked” aneurysms are not generally amenable to this type of treatment due to the likelihood that the coil(s), once positioned, will not be successfully retained within the aneurysm sac. Another limitation associated with the common single thread coil configuration is the number of manipulations frequently required in order for the surgeon to introduce a sufficient length of the coil within the aneurysm and the increased risk associated with such manipulations.

In an effort to improve the retention of coils in aneurysms exhibiting such wide-necked anatomy, intracranial stents have been developed for placement in the parent blood vessel to act as a buttress for holding the coil(s) in place within the aneurysmal sac. This approach, however, necessitates the placement of one or more permanent stents in the blood vessels of the brain. The use of permanent intracranial stents have been associated with increased morbidity in both the short term (adverse effects incurred during placement) as well as the long term (post-operative intracranial stenosis).

It would be desirable to provide a self-expandable aneurysm filling device, system and method that can not only cover the neck of an aneurysm, but that can also serve as a permanent embolic plug in the aneurysm. It would be desirable to provide a self-expandable aneurysm filling device, system and method that also achieves a generally spherical configuration using a single unified complex matrix that can be deposited inside an aneurysm for treatment of an aneurysm, to avoid the need to manipulate or move the self-expandable aneurysm filling device for implantation in the aneurysm. It would also be desirable to provide a self-expandable aneurysm filling device, system and method that can be used as the sole mechanical stabilization for an aneurysm, or that can serve as an anchor for holding other coils, glue or other compositions within an aneurysm. The present invention meets these and other needs.

### SUMMARY OF THE INVENTION

Briefly, and in general terms, the present invention provides for a self-expandable aneurysm filling device for treatment of an aneurysm, and a system and method for deploying the self-expandable aneurysm filling device into the aneurysm from a parent vessel for treatment of the aneurysm to at least partially fill and stabilize the aneurysm. In one aspect, the system provides a self-expandable aneurysm filling device that can cover the neck of an aneurysm, and can act as a permanent embolic plug in the aneurysm. The self-expandable aneurysm filling device also provides a single unified complex matrix that expands as it is deployed and achieves a generally spherical or ovoid configuration, so that the self-expandable aneurysm filling device does not need to be manipulated in the aneurysm. The self-expandable aneurysm filling device can be used to independently mechanically stabilize an aneurysm, or be used as an anchor for other coils, glue or other compositions.

Accordingly, the present invention provides for a self-expandable aneurysm filling system that includes a self-expandable aneurysm filling device having a compressed undeployed configuration and an expanded three-dimensional deployed configuration, a pusher wire and a severable deployment junction releasably connecting the self-expandable aneurysm filling device to the pusher wire. In a presently preferred aspect, the deployed configuration of the self-expandable aneurysm filling device is generally spherical or ovoid. In one embodiment, at least a portion of the self-expandable aneurysm filling device is formed from a shape memory material, such as nitinol. In another embodiment, the self-expandable aneurysm filling device is constructed of a metal such as platinum or platinum alloys. The severable deployment junction may be mechanically, electrolytically, or thermally severed to separate the self-expandable aneurysm filling device from the pusher wire. In a presently preferred aspect, the severable deployment junction is capable of being severed by electrical current, and an attachment fixture is provided for applying electrical current to the severable deployment junction to sever the severable deployment junction.

In the system and method of the invention, a microcatheter can also be provided for delivering the self-expandable aneurysm filling device in the compressed configuration into an aneurysm for treatment of the aneurysm. The self-expandable aneurysm filling device is delivered in the compressed configuration in an undeployed state through the microcatheter. The microcatheter is inserted inside the aneurysm, and the self-expandable aneurysm filling device is pushed through the microcatheter with the pusher wire until the self-expandable aneurysm filling device exits the microcatheter and deploys as a single unit into the aneurysm. As the self-expandable aneurysm filling device exits the microcatheter, it transforms from the compressed configuration into an expanded configuration, and thereby is allowed to expand within the aneurysm to achieve a completely deployed state, after which the self-expandable aneurysm filling device is separated from the pusher wire, and the microcatheter and pusher wire are withdrawn from the parent blood vessel. The step of separating the self-expandable aneurysm filling device from the pusher wire can be carried out by mechanically, thermally, or electrolytically severing the severable deployment junction to separate the self-expandable aneurysm filling device from the pusher wire. In a presently preferred aspect of the invention, the severable deployment junction is capable of being severed by electrical current, and the step of separating the self-expandable aneurysm filling device from the pusher wire is carried out by applying electrical current to the severable junction to sever the severable junction.

These and other aspects and advantages of the invention will become apparent from the following detailed description and the accompanying drawings, which illustrate by way of example the features of the invention.

### BRIEF DESCRIPTION OF THE DRAWINGS

FIGS. 1A-1D illustrate the method of deployment of a self-expandable aneurysm filling device into an aneurysm according to the invention.

FIG. 2 is a schematic diagram illustrating the basic apparatus of one example embodiment of a self-expandable aneurysm filling system including a self-expandable aneurysm filling device shown in an expanded configuration and connected to a pusher wire, according to the present invention.

FIG. 3 is a schematic diagram illustrating a collapsed or compressed configuration of the self-expandable aneurysm filling device of FIG. 2 that is suitable for use in practicing the invention positioned within a microcatheter.

FIGS. 4A-4C illustrates another embodiment of a method of deployment of a self-expandable aneurysm filling device into an aneurysm according to the invention.

FIG. 5 illustrates an example embodiment of self-expandable aneurysm filling device formed from an elongated strand of shape memory material shown in an expanded configuration and connected to a pusher wire.

FIG. 6 illustrates an example embodiment of a self-expandable aneurysm filling device formed from an elongated strand of shape memory material shown in an expanded configuration.

FIG. 7 illustrates an example embodiment of a self-expandable aneurysm filling device formed from an elongated strand of shape memory material shown in an expanded configuration.

FIG. 8 illustrates an example embodiment of a self-expandable aneurysm filling device formed from an elongated strand of shape memory material shown in an expanded configuration.

FIG. 9 illustrates an example embodiment of a self-expandable aneurysm filling device formed from an elongated strand of shape memory material shown in an expanded configuration.

FIG. 10a illustrates an example embodiment of a self-expandable aneurysm filling device having a four petal configuration formed from four elongated stands of a shape memory material shown in an expanded configuration.

FIG. 10b illustrates an alternate view of the self-expandable aneurysm filling device of FIG. 10a.

FIG. 11a illustrates an example embodiment of a self-expandable aneurysm filling device having a five petal configuration formed from five elongated strands of shape memory material shown in an expanded configuration.

FIG. 11b illustrates the self-expandable aneurysm filling device similar to the device of FIG. 11a having a four petal configuration formed from four elongated strands of a shape memory material and having a pusher wire connected thereto.

FIG. 11c illustrates an alternate view of self-expandable aneurysm filling device of FIG. 11b having a connected pusher wire.

FIG. 11d illustrates self-expandable aneurysm filling device of FIG. 11b with the pusher wire removed.

FIG. 12a illustrates an example embodiment of a self-expandable aneurysm filling device formed from six elongated strands of a shape memory material having a six-petal-atom configuration when expanded.

FIG. 12b illustrates an alternate view of the self-expandable aneurysm filling device of FIG. 12a.

FIG. 13a illustrates an example embodiment of a self-expandable aneurysm filling device formed from eight elongated strands of a shape memory material having an eight-petal-atom configuration when expanded.

FIG. 13b illustrates an alternate view of the self-expandable aneurysm filling device of FIG. 13a.

### DETAILED DESCRIPTION OF THE PREFERRED EMBODIMENTS

In one exemplary embodiment, the self-expandable aneurysm filling system of the present invention provides for a self-expandable aneurysm filling device that achieves a generally spherical configuration using a single unified complex matrix that is deposited inside the aneurysm to act as a stent. This design obviates the need to manipulate or move the stent being implanted, since the shape of the stent is predetermined so that the self-expandable aneurysm filling device deploys as a single complex sphere. Once deployed, the self-expandable aneurysm filling device acts as an anchor for holding other coils, glue or other compositions within the aneurysm sac.

A self-expandable aneurysm filling device according to the invention does not merely cover the neck of an aneurysm, but also acts as an embolic plug, and is permanent. Depending on the configuration and size of the aneurysm, as well as the preferences of the surgeon, self-expandable aneurysm filling devices according to the invention may also be used as the sole mechanical stabilization for an aneurysm.

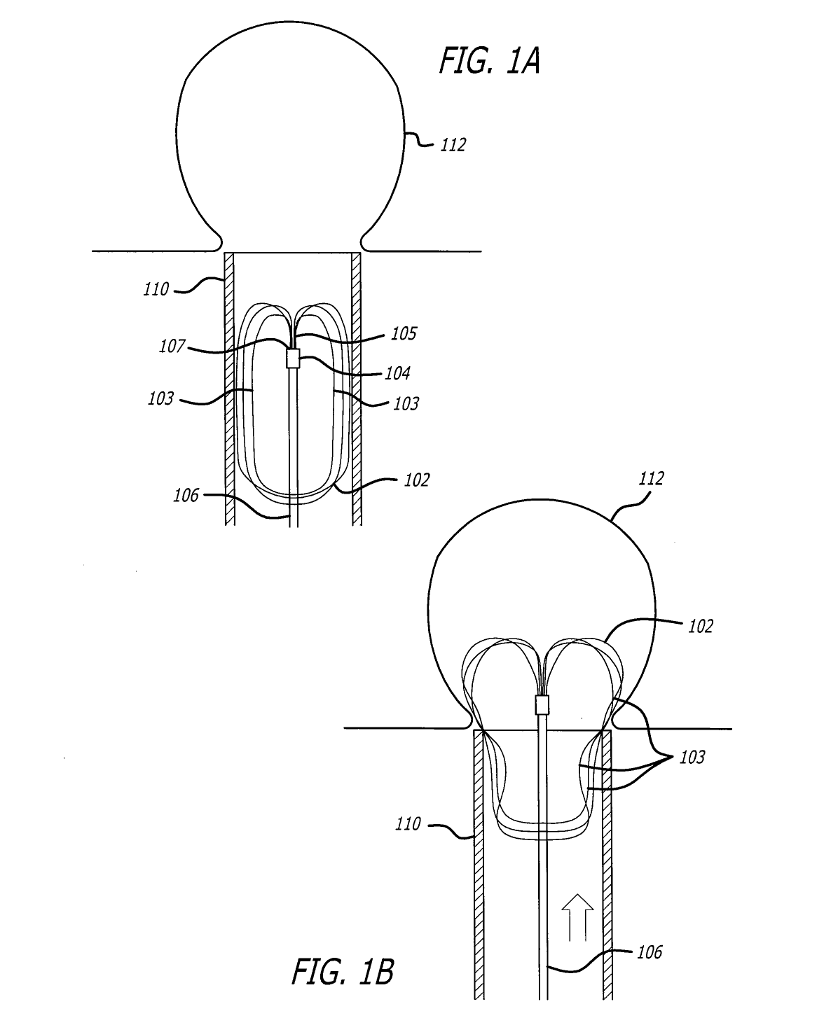

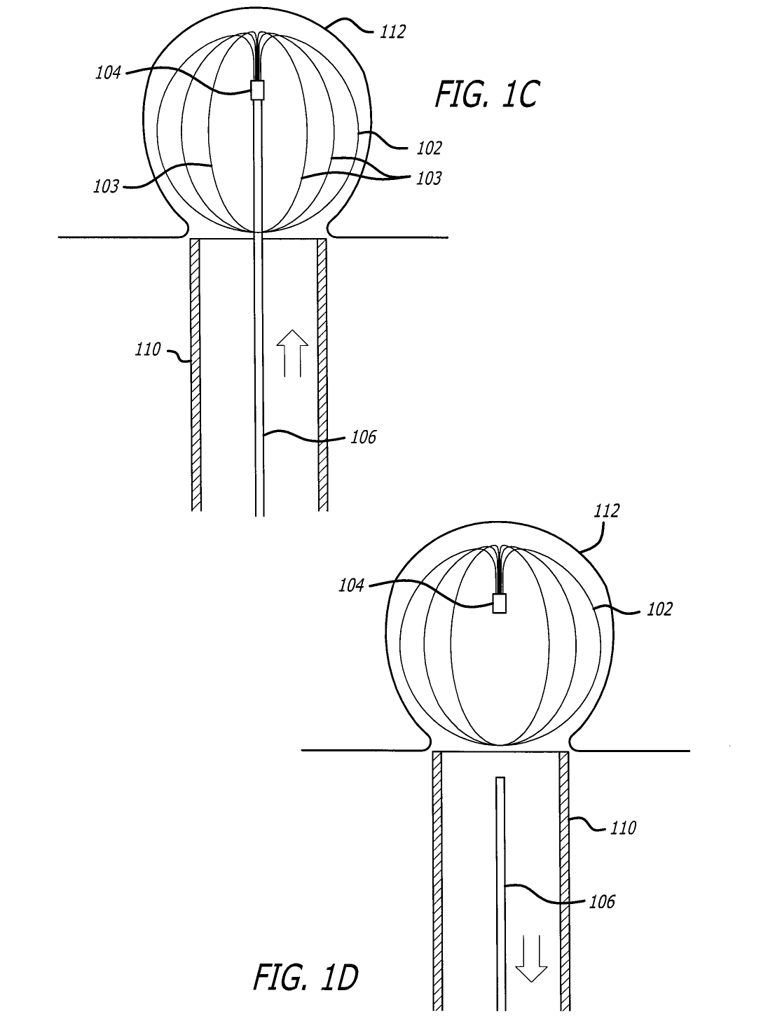

With reference to FIGS. 1A-1D, the present invention provides for a method of deployment of a self-expandable aneurysm filling device according to the invention into an aneurysm 112 extending from a primary or parent blood vessel (not shown). Referring to FIG. 1A, the basic apparatus of the self-expandable aneurysm filling system of the present invention includes a self-expandable aneurysm filling device 102, shown in a compressed configuration, connected by a severable joint or deployment junction 104 to a pusher wire 106. The pusher wire may also provide one or more attachment fixtures (not shown) for applying electrical current from an external power supply (not shown). The self-expandable aneurysm filling device is preferably formed from a plurality of elongated strands of shape memory material 103 having connection ends 105 connected together at an attachment location 107. In one embodiment, the plurality of elongated strands 103 are formed from nitinol. Further, as is illustrated, the self-expandable aneurysm filling device 102 may be positioned within a microcatheter 110 in a collapsed or compressed form.

The self-expandable aneurysm filling device may be delivered in a collapsed or compressed configuration in an undeployed state 102 through the microcatheter 110 to the site of an aneurysm 112. As self-expandable aneurysm filling device 102 is deployed and exits the microcatheter it transforms from its compressed state to an expanded state, as illustrated in FIG. 1B. Once self-expandable aneurysm filling device 102 has been fully deployed into the aneurysm 112 it is able to fully expand as shown in FIG. 1C. Once the self-expandable aneurysm filling device 102 has been fully deployed, the deployment junction 104 is activated to release the self-expandable aneurysm filling device 102 and to allow the microcatheter 110 and pusher wire 106 to be withdrawn from the parent blood vessel, as illustrated in FIG. 1D.

It should be noted that these figures are intended to illustrate the general characteristics of methods and materials with reference to certain example embodiments of the invention and thereby supplement the detailed written description provided below. These drawings are not, however, to scale and may not precisely reflect the characteristics of any given embodiment, and should not be interpreted as defining or limiting the range of values or properties of embodiments within the scope of this invention. In particular, the relative sizing and positioning of particular elements and structures may be reduced or exaggerated for clarity. The use of similar or identical reference numbers in the various drawings is intended to indicate the presence of a similar or identical element or feature.

In one embodiment, the self-expandable aneurysm filling devices according to the invention are preferably constructed of platinum and its alloys in order to take advantage of the properties of these materials with respect to their ability to retain memory for shape, resistance to biological fluids, softness and non-ferromagnetic properties that will allow patients to undergo MRI procedures and pass through metal detectors. Although platinum and its alloys are preferred, those skilled in the art will appreciate that other materials and, in some instances, combinations of two or more materials including, for example, other metals and polymers, may be utilized for constructing self-expandable aneurysm filling devices according to the invention. Optionally, in an alternate embodiment, at least a portion of self-expandable aneurysm filling device 102 is formed of a super-elastic material. Alternately, in another preferred embodiment, at least a portion of self-expandable aneurysm filling device 102 is formed from a shape memory material. In one embodiment, the shape memory material is nitinol. Regardless of the material or materials used in constructing the self-expandable aneurysm filling devices 102, it will be characterized by a deployed configuration that is generally spherical, ovoid or otherwise shaped to avoid the loose ends associated with conventional coils filling an aneurysm.

The self-expandable aneurysm filling devices 102 according to the invention may be introduced through a microcatheter 110 that is placed inside the aneurysm 112 as is standard for current treatment. The self-expandable aneurysm filling device 102 is then pushed through the microcatheter 110 with a thin wire, often referred to as a pusher wire 106, until it exits the microcatheter and deploys as a single unit into the aneurysm sac under direct fluoroscopic observation. Referring back to FIG. 1B, as the self-expandable aneurysm filling device 102 deploys from within the microcatheter 110, it assumes, or is induced to assume, its full 3-dimensional configuration and, when appropriately sized for the aneurysm under treatment, the size of the self-expandable aneurysm filling device will exceed the opening from the aneurysm into the parent vessel and will thereby be retained indefinitely within the aneurysm sac exhibit and will not present any loose ends that would extend out of the aneurysm.

In one optional embodiment, the self-expandable aneurysm filling device 102 may be retracted back into the microcatheter 110 during the deployment process by pulling the pusher wire 106 back into the microcatheter 110 thereby causing the self-expandable aneurysm filling device 102 to follow. During retraction, the self-expandable aneurysm filling device 102 will transform back into a compressed configuration to enter the microcatheter 110.

Referring back to the process of deploying the self-expandable aneurysm filling device 102 through the microcathether 110 out into the aneurysm 110, once adequate deployment is achieved, i.e., when the self-expandable aneurysm filling device has been completely ejected from the delivery microcatheter 110 and is satisfactorily positioned within the aneurysm sac, the self-expandable aneurysm filling device 102 may be separated from its feed wire through the electrolytic or thermal means. The feed wire (or pusher wire 106) may then be withdrawn through the microcatheter 110 and discarded while leaving the self-expandable aneurysm filling device 102 in place.

The disclosed delivery system provides means for introducing a generally spherical or ovoid device within the aneurysm sac to at least partially fill and stabilize the aneurysm under treatment. The self-expandable aneurysm filling device 102 may be used alone or may be used in combination with other vaso-occlusive devices, including conventional coils, and/or materials, including materials intended to promote and/or suppress certain effects and responses within the aneurysm and the surrounding tissue. A variety of coatings and compositions have, for example, been proposed for suppressing intimal thickening by reducing the stimulus resulting from placement of the stent and the associated thrombosis or restenosis. Other coatings and compositions may be included, singly or in combination, for delivering one or more pharmaceutical/therapeutic agents to retard smooth muscle tissue proliferation or restenosis.

As noted above, the self-expandable aneurysm filling device 102 will typically be attached to the distal end of a feed, guide, pusher or core wire that can then be used to guide the device through a microcatheter into the aneurysm. A severable joint, also referred to as a deployment junction 104, will typically be provided at the junction of the feed wire 106 and the device 102 for separating after deployment within the aneurysm sac. As known to those skilled in the art, a variety of severable joints have been utilized in such applications to provide for mechanical, electrolytic and thermal separation of the pusher wire and the stent assembly.

A variety of mechanically detachable devices are known to those in the art including, for example, embodiments in which a helically wound coil may be unscrewed from a pusher wire providing an interlocking surface, releasing interlocking clasps or other complementary structures provided on distal end of the pusher wire and the coil respectively. The interlocking surface on the self-expandable aneurysm filling device may be provided externally or internally on the device structure. Other more complex mechanisms which employ additional structures including, for example, a pusher sheath, have also been utilized for releasing the device from the pusher wire.

In contrast to the mechanical release mechanisms, electrolytically severable joints are severed by application of an appropriate voltage on the core wire and thereby induce a current through the joint. The joint erodes in preference either to the vaso-occlusive device or to the pusher wire. Utilizing the principles of competitive erosion, those portions of the wire and device apart from the joint region that are not intended to erode may be insulated to suppress any electrolytic response. In addition to the mechanical and electrolytic severable joints, thermal joints release under the application of heating, typically resistance heating resulting from an electrical current flowing through the joint to weaken and/or melt the joint material to a degree sufficient to release the device from the pusher wire.

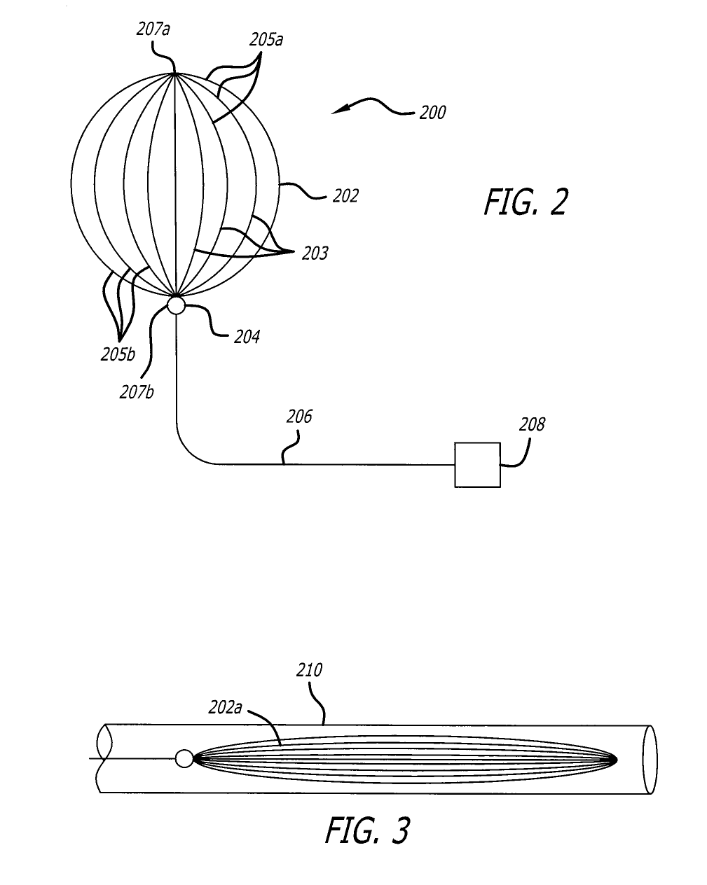

FIG. 2 illustrates an alternate embodiment of a self-expandable aneurysm filling system. Referring to FIG. 2, the basic apparatus 200 of the self-expandable aneurysm filling system includes a self-expandable aneurysm filling device 202, shown in an expanded configuration, connected by a severable joint or deployment junction 204 to a pusher wire 206. The pusher wire may also provide one or more attachment fixtures 208 for applying electrical current from an external power supply (not shown). The self-expandable aneurysm filling device is preferably formed from a plurality of elongated strands of shape memory material 203 having first ends 205a connected together at a first attachment location 207a, and second ends 205b connected together at a second attachment location 207b. As is illustrated in FIG. 3, in a collapsed or compressed configuration, the self-expandable aneurysm filling device 202a may be positioned within a microcatheter 210 that is suitable for use in practicing the invention.

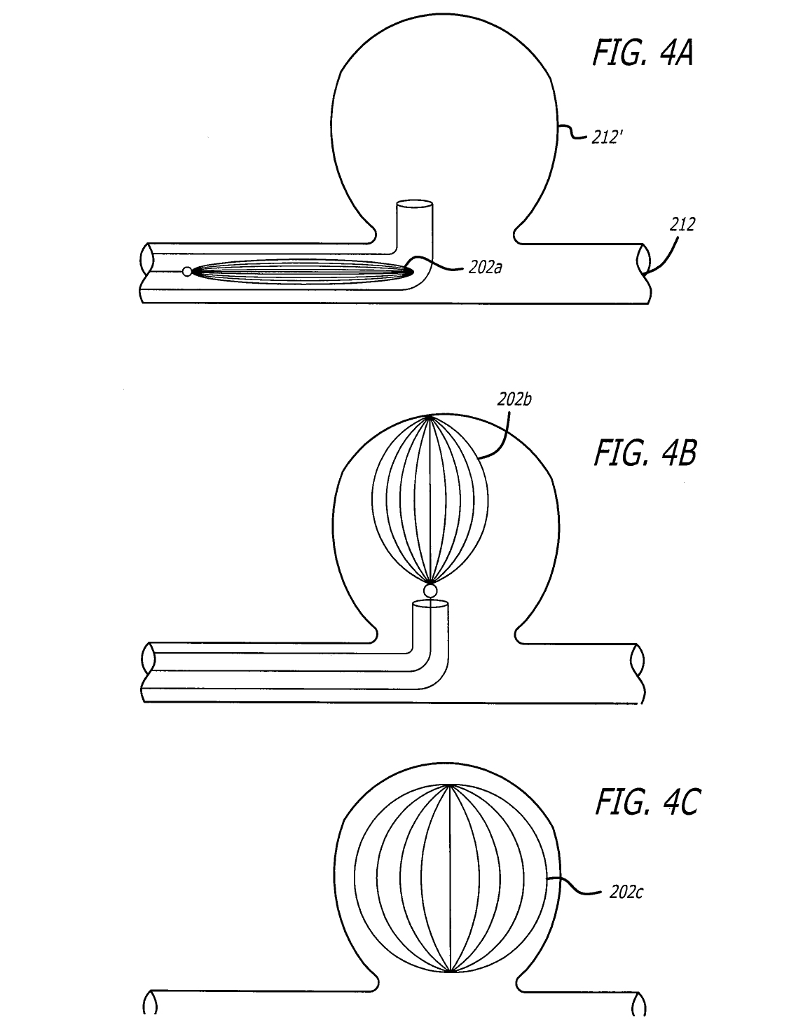

With reference to FIGS. 4A-4C, the present invention also provides for an alternate embodiment of a method of deployment of a self-expandable aneurysm filling device into an aneurysm 212′ extending from a primary or parent blood vessel 212. The self-expandable aneurysm filling device may be delivered in a collapsed or compressed configuration in an undeployed state 202 through the microcatheter 210 to the site of an aneurysm 212, and then deployed into the aneurysm in a partially deployed state 202b by the microcatheter, as illustrated in FIG. 4B, and, finally, the self-expandable aneurysm filling device self-expands within the aneurysm to achieve a completely deployed state 202c, as shown in FIG. 4C, after which the deployment junction is activated to release the self-expandable aneurysm filling device and to allow the microcatheter and pusher wire to be withdrawn from the parent blood vessel.

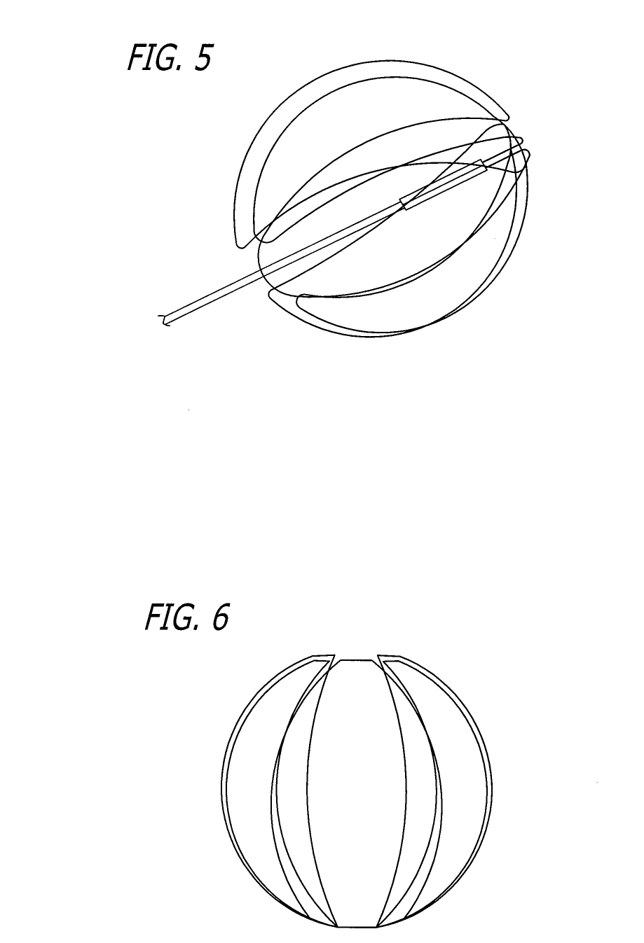

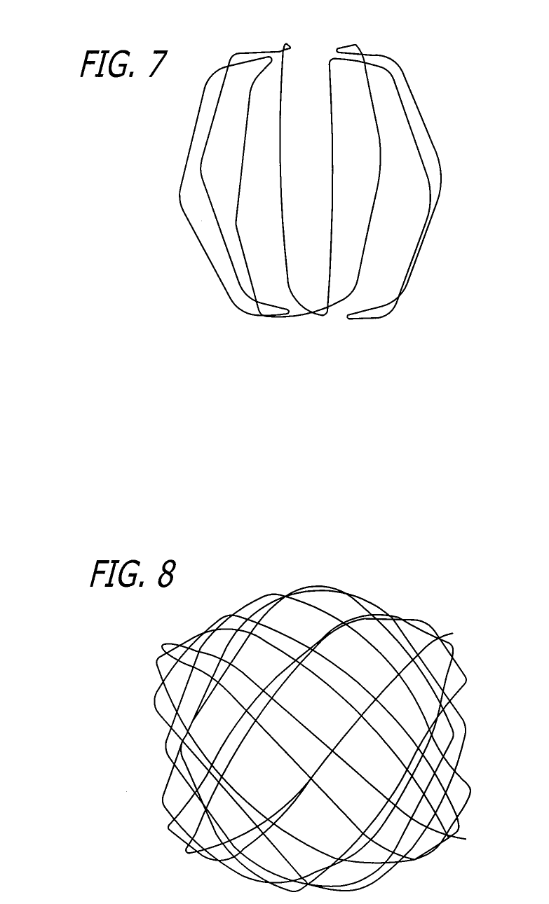

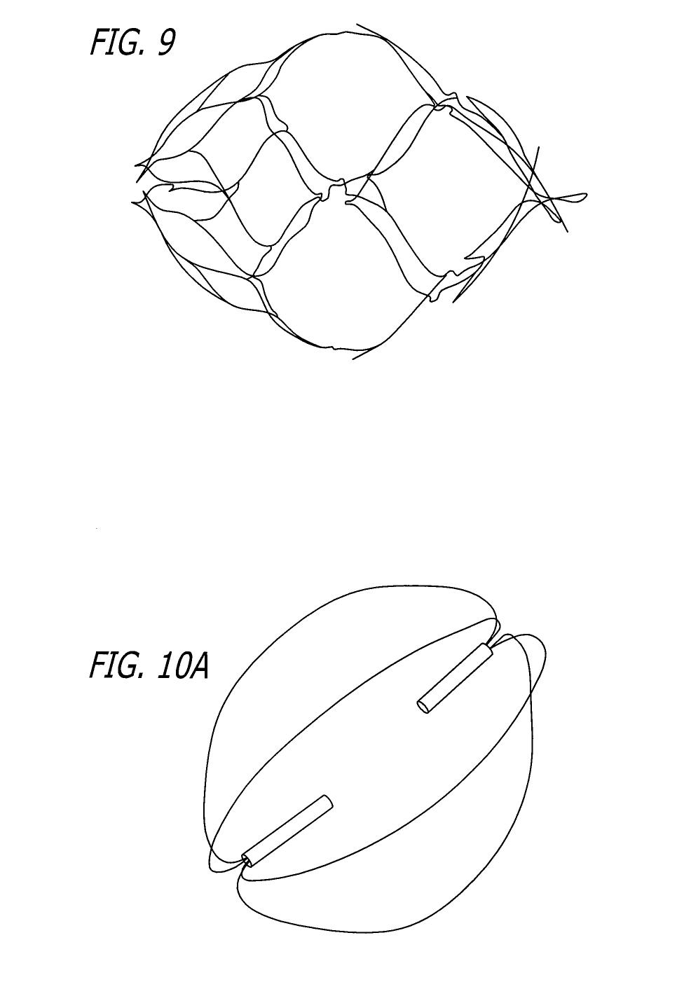

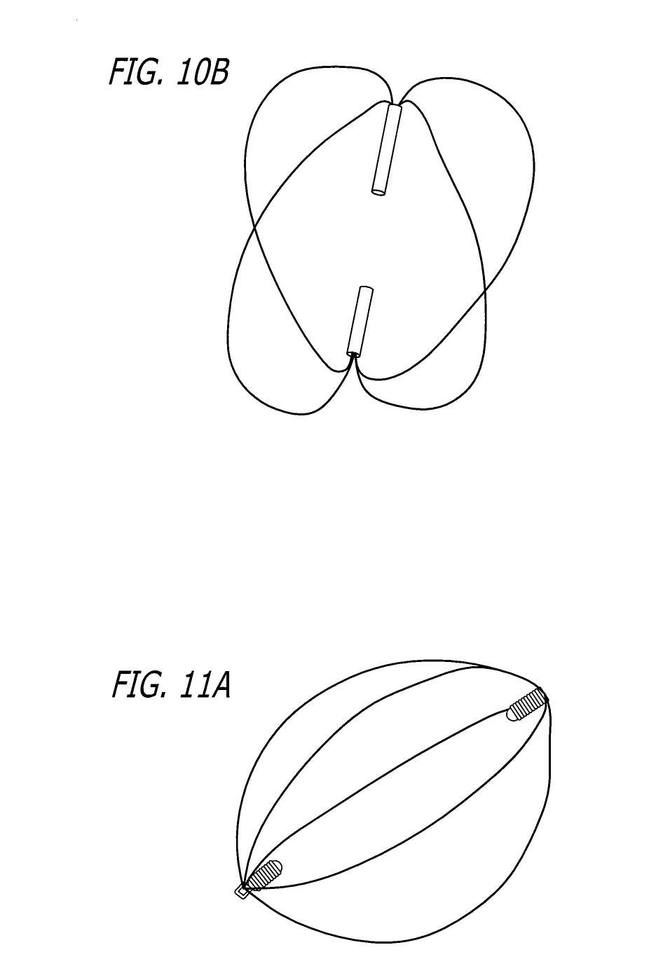

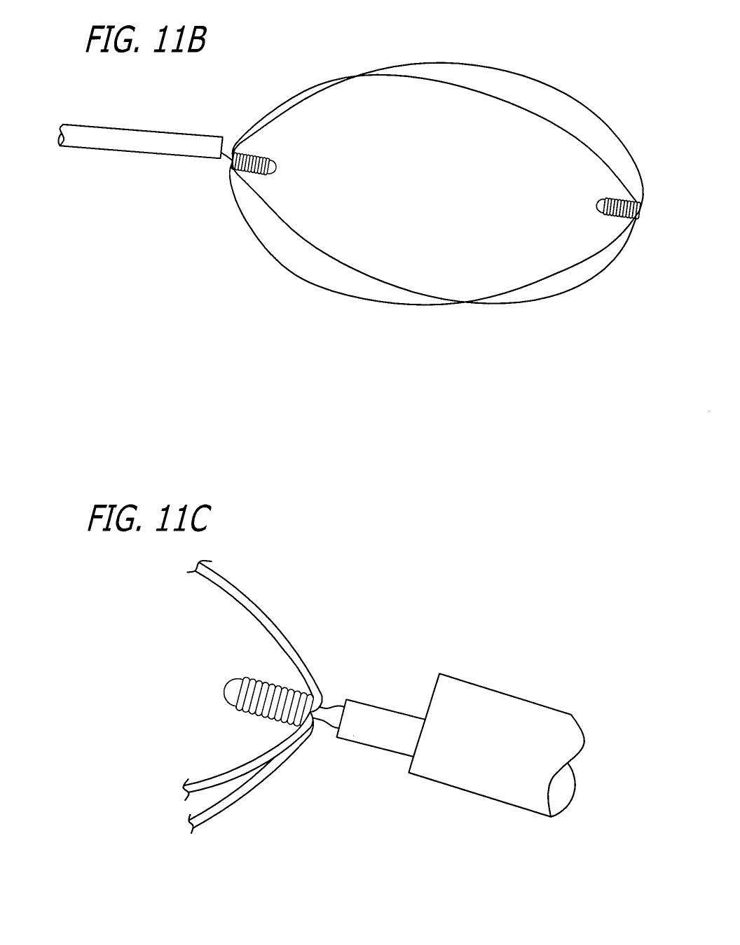

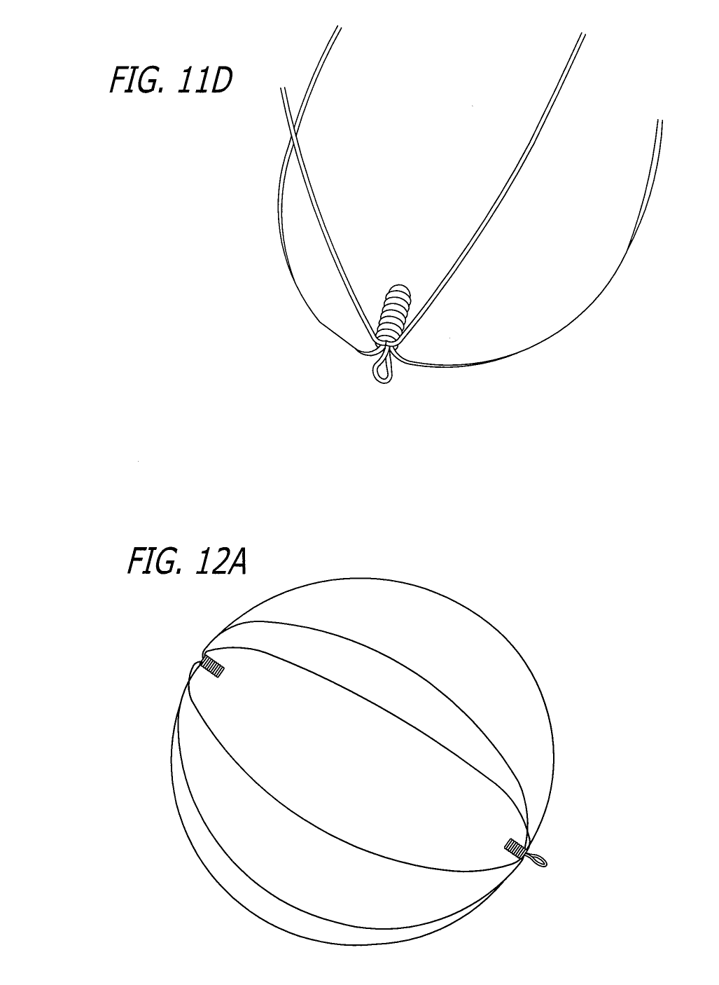

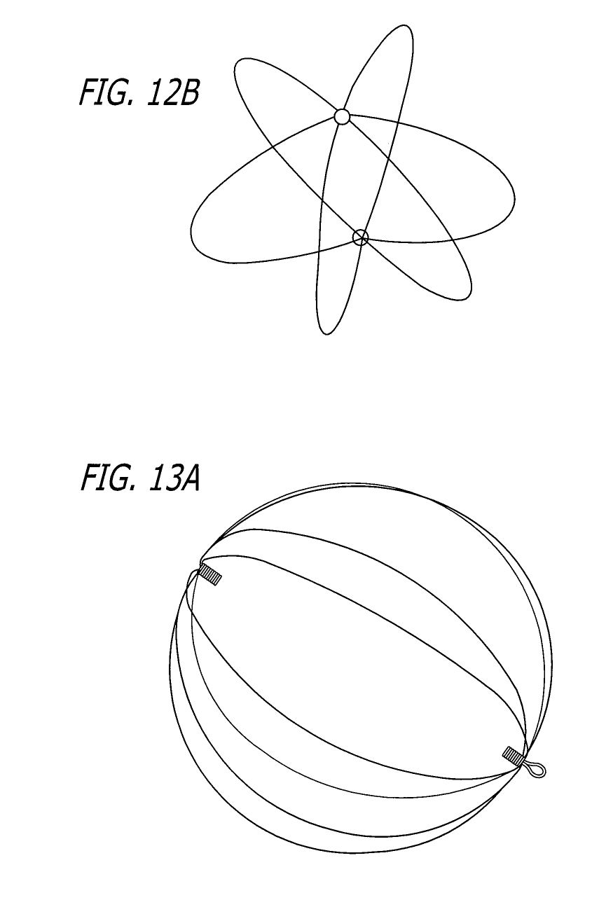

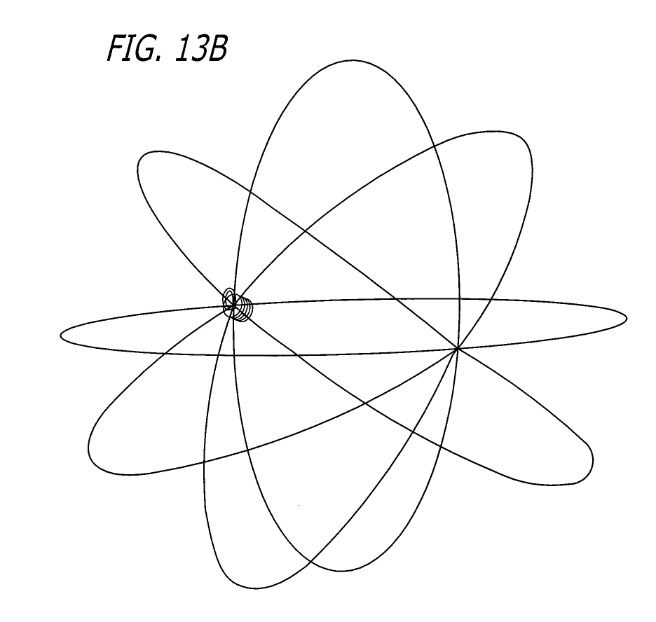

With references to FIGS. 5-13B, the present invention also provides for alternate shapes and configurations of a self-expandable aneurysm filling device formed of one or more elongated strands of shape memory material connectable together at an attachment location and deliverable from a deployment junction as described in the foregoing embodiments. As is illustrated in FIGS. 10a to 13b, the self-expandable aneurysm filling devices may be formed with multiple elongated strands of a shape memory material forming space-filling cages with a corresponding number of segments, lobes, petals or ribs for example. The self-expandable aneurysm filling devices shown may be built by winding nitinol wire around a mandrel in a particular winding pattern. The mandrel with wire windings is then heated in a furnace for a set amount of time. After heating, the mandrel is quenched with coolant and thus cooled. Compressed air may be used to remove excess coolant from the mandrel. The wire is cut and removed from the mandrel.

A variety of designs, materials and procedures have been disclosed in other publications including, for example, U.S. Patent Application Nos. 2007/0150045; 2007/0106311; 2007/0036042; 2006//0206199; 2006/0155323; 2006/0106421; 2005/0251200; 2005/0249776; 2005/0033409; 2004/0193246; 2004/0193206; 2004/0098027; 2004/0093014; 2004/0044391; 2003/0181927; 2003/0171739; 2003/0083676; 2003/0028209; 2003/0018294; 2003/0004681; 2001/0007946; and U.S. Pat. Nos. 7,241,301; 7,232,461; 7,201,762; 7,195,636; 7,128,736; 6,953,472; 6,936,055; 6,855,153; 6,811,560; 6,802,851; 6,793,664; 6,723,112; 6,645,167; 6,592,605; 6,589,265; 6,585,748; 6,569,179; 6,540,657; 6,511,468; 6,506,204; 6,454,780; 6,383,174; 6,344,041; 6,299,619; 6,238,403; 6,231,590; 6,193,708; 6,187,024; 6,183,495; 6,171,326; 6,168,615; 6,186,592; 6,139,564; 6,096,034; 6,093,199; 6,090,125; 6,086,577; 6,063,104; 6,063,070; 6,036,720; 5,980,554; 5,980,514; 5,935,148 and 5,108,407; the contents of each publication being incorporated herein in its entirety.

It will be apparent from the foregoing that while particular forms of the invention have been illustrated and described, various modifications can be made without departing from the spirit and scope of the invention. Accordingly, it is not intended that the invention be limited, except as by the appended claims.

What is claimed is:

1. An aneurysm filling apparatus for treatment of an aneurysm, comprising:
a self-expandable aneurysm filling device having a compressed undeployed configuration and an expanded three-dimensional deployed configuration, wherein the self-expandable aneurysm filling device transforms from the compressed configuration to the expanded three-dimensional configuration as it is deployed;
a pusher wire; and
a severable deployment junction releasably connecting said self-expandable aneurysm filling device to said pusher wire.

2. The aneurysm filling apparatus of claim 1, wherein said deployed configuration of said self-expandable aneurysm filling device is generally spherical.

3. The aneurysm filling apparatus of claim 1, wherein said deployed configuration of said self-expandable aneurysm filling device is generally ovoid.

4. The aneurysm filling apparatus of claim 1, wherein said self-expandable aneurysm filling device comprises a metal selected from the group consisting of platinum and platinum alloys.

5. The aneurysm filling apparatus of claim 1, wherein at least a portion of the self-expandable aneurysm filling device is formed of a super-elastic material.

6. The aneurysm filling apparatus of claim 1, wherein at least a portion of the self-expandable aneurysm filling device is formed from a shape memory material.

7. The aneurysm filling apparatus of claim 6, wherein the shape memory material is nitinol.

8. The aneurysm filling apparatus of claim 1, wherein said severable deployment junction comprises means for mechanically severing the self-expandable aneurysm filling device from said pusher wire.

9. The aneurysm filling apparatus of claim 1, wherein said severable deployment junction comprises means for electrolytically severing the self-expandable aneurysm filling device from said pusher wire.

10. The aneurysm filling apparatus of claim 1, wherein said severable deployment junction comprises means for thermally severing the self-expandable aneurysm filling device from said pusher wire.

11. The aneurysm filling apparatus of claim 1, wherein said severable deployment junction is capable of being severed by electrical current, and further comprising an attachment fixture for applying electrical current to the severable deployment junction to sever the severable deployment junction.

12. A self-expandable aneurysm filling system for deploying a self-expandable aneurysm filling device into an aneurysm from a parent vessel for treatment of the aneurysm to at least partially fill and stabilize the aneurysm, comprising:
a self-expandable aneurysm filling device having a compressed undeployed configuration and an expanded three-dimensional deployed configuration;
a pusher wire;
a severable deployment junction releasably connecting said self-expandable aneurysm filling device to said pusher wire; and
a microcatheter for delivering the self-expandable aneurysm filling device in the compressed configuration into an aneurysm for treatment of the aneurysm, wherein the self-expandable aneurysm filling device transforms from the compressed configuration to the expanded configuration as it is deployed through the microcatheter.

13. The self-expandable aneurysm filling system of claim 12, wherein said deployed configuration of said self-expandable aneurysm filling device is generally spherical.

14. The self-expandable aneurysm filling system of claim 12, wherein said deployed configuration of said self-expandable aneurysm filling device is generally ovoid.

15. The self-expandable aneurysm filling system of claim 12, wherein said self-expandable aneurysm filling device is constructed of a metal selected from the group consisting of platinum and platinum alloys.

16. The aneurysm filling apparatus of claim 12, wherein at least a portion of the self-expandable aneurysm filling device is formed of a super-elastic material.

17. The aneurysm filling apparatus of claim 12, wherein at least a portion of the self-expandable aneurysm filling device is formed from a shape memory material.

18. The aneurysm filling apparatus of claim 17, wherein the shape memory material is nitinol.

19. The self-expandable aneurysm filling system of claim 12, wherein said severable deployment junction comprises means for mechanically severing the self-expandable aneurysm filling device from said pusher wire.

20. The self-expandable aneurysm filling system of claim 12, wherein said severable deployment junction comprises means for electrolytically severing the self-expandable aneurysm filling device from said pusher wire.

21. The self-expandable aneurysm filling system of claim 12, wherein said severable deployment junction comprises means for thermally severing the self-expandable aneurysm filling device from said pusher wire.

22. The self-expandable aneurysm filling system of claim 12, wherein said severable deployment junction is capable of being severed by electrical current, and further comprising an attachment fixture for applying electrical current to the severable deployment junction to sever the severable deployment junction.

23. A method of deploying a self-expandable aneurysm filling device into an aneurysm from a parent vessel for treatment of the aneurysm to at least partially fill and stabilize the aneurysm, comprising the steps of:
providing a self-expandable aneurysm filling device having a compressed undeployed configuration and an expanded three-dimensional deployed configuration, a pusher wire, a severable deployment junction releasably connecting said self-expandable aneurysm filling device to said pusher wire, and a microcatheter for delivering the self-expandable aneurysm filling device in the compressed configuration into an aneurysm for treatment of the aneurysm;
delivering the self-expandable aneurysm filling device in the compressed configuration in an undeployed state through the microcatheter to an aneurysm;
inserting a distal portion of the microcatheter inside the aneurysm;
pushing the self-expandable aneurysm filling device through the microcatheter with the pusher wire until the self-expandable aneurysm filling device exits the microcatheter, wherein the self-expandable aneurysm filling device transforms from the compressed configuration into the expanded configuration as it exits through the microcathether;
allowing the self-expandable aneurysm filling device to expand within the aneurysm to achieve a completely deployed state;
separating the self-expandable aneurysm filling device from the pusher wire; and
withdrawing the microcatheter and pusher wire from the parent blood vessel.

24. The method of claim 23, wherein said step of separating the self-expandable aneurysm filling device from the pusher wire comprises mechanically severing the severable deployment junction to separate said self-expandable aneurysm filling device from said pusher wire.

25. The method of claim 23, wherein said step of separating the self-expandable aneurysm filling device from the pusher wire comprises thermally severing the severable deployment junction to separate said self-expandable aneurysm filling device from said pusher wire.

26. The method of claim 23, wherein the severable deployment junction is capable of being severed by electrical current, and said step of separating the self-expandable aneurysm filling device from the pusher wire comprises applying electrical current to the severable junction to sever the severable junction.
